(19) United States
(12) Patent Application Publication
(10) Pub. No.: US 2004/0137028 A1
(43) Pub. Date: Jul. 15, 2004

(54) Transfer-free cosmetic composition comprising a dispersion of polymer particles and a specific rheological agent

(75) Inventor: Valerie de la Poterie, Le Chatelet En Brie (FR)

Correspondence Address: FINNEGAN, HENDERSON, FARABOW, GARRETT & DUNNER, LLP, 1300 I STREET, NW, WASHINGTON, DC, 20005, US

(73) Assignee: L'OREAL

(21) Appl. No.: 10/750,765

(22) Filed: Jan. 5, 2004

Related U.S. Application Data
(63) Continuation of application No. 09/437,109, filed on Nov. 9, 1999, now abandoned.

(30) Foreign Application Priority Data
Nov. 9, 1998 (FR) 98 14076

(51) Int. Cl. A61K 7/06; A61K 7/11

(52) U.S. Cl. 424/401; 424/70.16

(57) ABSTRACT

The present invention relates to a composition and a process for making and using the same, especially a cosmetic, dermatological, hygiene or pharmaceutical composition, for caring for and/or making up the skin, superficial body growths and lips, having transfer-free properties while being very comfortable, which can be provided in the form of a cast product or of a gel having a dispersion of polymer particles which are stabilized at the surface in a liquid fatty phase by a stabilizing agent, the fatty phase furthermore being thickened by a fat-soluble rheological agent resulting from the polymerization of at least one monomer possessing (an) ethylenic bond(s).

The present invention relates to a composition comprising at least one polymer that is dispersible in a fatty phase, which composition is intended, in particular, for the cosmetic, dermatological, pharmaceutical and hygiene fields. More specifically, the invention relates to a transfer-free composition for caring for and/or making up the skin, both of the human face and of the human body, mucous membranes, such as the lips and the interior of the lower eyelids, and superficial body growths, such as the eyelashes, eyebrows, nails and hair.

This composition can be provided in the form of a product cast as a stick or as a disk, such as lipsticks or lip balms, cast foundations, products for concealing rings under the eyes, eyeshadows or blushers, or in the form of a more or less fluid paste or cream, such as fluid foundations or lipsticks, eyeliners, mascaras, solar protection compositions or compositions for coloring the skin or for making up the body.

The known products for making up or caring for the skin or lips of human beings, such as foundations or lipsticks, generally comprise fatty phases, such as waxes and oils, pigments and/or fillers and, optionally, additives, such as cosmetic or dermatological active principles. They can also comprise so-called “pasty” products with a supple consistency which make it possible to produce colored or non-colored pastes for application with a brush.

These known compositions, when applied to the skin or the lips, exhibit the disadvantage of transferring, i.e., being at least partly deposited and leaving traces on certain substrates they are brought into contact with, for example, a glass, a cup, a cigarette, an item of clothing or the skin. This deposit results in mediocre persistence of the applied film, requiring regular renewal of the application of the foundation or lipstick composition. Furthermore, the appearance of these unacceptable traces, particularly on blouse collars, can dissuade some women from using this type of make-up.

Furthermore, these compositions have a tendency to migrate, i.e., to spread inside the wrinkles and fine lines of the skin which surround the lips and eyes, producing an unattractive effect.

In Japanese Patent Application JP-A-61-65809, the company Shiseido has disclosed “transfer-free” lipstick compositions comprising a siloxysilicate resin having a three-dimensional network, a volatile silicone oil with a cyclic silicone chain, and pulverulent fillers. Furthermore, the company Noevier has disclosed, in Japanese Patent Application JP-A-62-61911, “transfer-free” lipstick, eyeliner and foundation compositions comprising one or more volatile silicones in combination with one or more hydrocarbon-comprising waxes.

These compositions, although exhibiting improved “transfer-free” properties, have the disadvantage of leaving on the lips, after evaporation of the silicone oils, a film which becomes uncomfortable over time (feeling of drying and of tautness), dissuading a number of women from this type of lipstick.

More recently, the company Procter & Gamble has envisaged, in its International Patent Application WO-A-96/36323, mascara compositions of water-in-oil emulsion type which exhibit a lengthy hold and resistance to water and which do not leave traces. These compositions comprise, inter alia, a water-insoluble polymer, generally known as a latex, in combination with a surfactant of the alkyl or alkoxy dimethicone copolyol type, hydrocarbon-comprising oils, pigments and fillers, as well as waxes.

Compositions based on silicone oils and silicone resins, as well as those based on latex, result in matte colored films. Women today are looking for glossy products, especially for coloring the lips. Furthermore, the transfer-free properties of the films deposited are not perfect. In particular, pronounced pressure or rubbing results in a decrease in the color of the deposit and in a redeposition on the substrate brought into contact with these films.

In addition, European Patent Application EP-A-497,144 and French Patent Application FR-A-2,357,244 disclose so-called “transfer-free” compositions comprising a styrene-ethylene-propylene block polymer in combination with waxes, light or volatile oils, and pigments. These compositions exhibit the disadvantage of being not very comfortable, of having indifferent cosmetic properties and of being difficult to formulate. Furthermore, the “transfer-free” properties of these compositions are mediocre.

The need thus remains for a composition which does not exhibit the above disadvantages and which has complete “transfer-free” properties, even during pronounced pressure or rubbing, and a more or less glossy appearance, suited to the wish of the consumer, which does not migrate and which does not dry the skin or the lips on which it is applied, either during application or over time.

The Inventor has found, entirely surprisingly, that the use of at least one polymer which is dispersible in a fatty phase, in combination with a specific rheological agent, in a cosmetic, dermatological, pharmaceutical or hygiene composition made it possible to obtain a glossy film, with very good hold, which transfers very little or not at all, which does not migrate and which is resistant to water, while being very pleasant on application and to wear throughout the day. The film is supple and flexible.

The subject of the present invention is thus a composition for topical application comprising a liquid fatty phase and polymer particles dispersed in the liquid fatty phase and stabilized at the surface by a stabilizing agent, the fatty phase additionally being thickened by a fat-soluble rheological agent resulting from the polymerization of at least one monomer possessing an ethylenic bond.

The phrase “monomer possessing an ethylenic bond” must be understood as meaning a monomer comprising one or more ethylenic bonds which are optionally conjugated.

This composition is, in particular, a cosmetic, dermatological, hygiene or pharmaceutical composition. It thus comprises ingredients which are compatible with the skin, mucous membranes and keratinous fibres or superficial body growths. It can be provided in the form of an anhydrous gel, of an oil-in-water or water-in-oil emulsion or dispersion, or in the form of a multiple emulsion.

The polymers that can be used in the present invention can be of any nature. Thus, it is possible to employ a radical polymer, a polycondensate, even a polymer of natural origin, and their mixtures. The polymer can be chosen by a person skilled in the art as a function of its properties and according to the subsequent application desired for the composition. The polymer used can preferably form a film. However, it is possible to use a polymer which cannot form a film.

The term “polymer which cannot form a film” is understood to mean a polymer which is not capable, alone, of forming an isolatable film. This polymer makes it possible, in combination with a non-volatile compound of the oil type, to form a continuous and homogeneous deposit on the skin and mucous membranes.

The composition advantageously comprises at least one ingredient chosen from cosmetic, dermatological, hygiene and pharmaceutical active principles, coloring materials and their mixtures. By virtue of the dispersion of polymer particles which are stabilized at the surface present in the liquid fatty phase, the composition of the invention makes it possible to limit, indeed even eliminate, the transfer of the composition and, in particular, the transfer of the active principles and/or coloring materials and thus to keep these active principles and/or coloring materials at the site where they were applied.

According to the invention, the amount of polymer must be sufficient to form, on the skin and/or lips and/or keratinous fibres, a film capable of trapping the coloring materials and/or a cosmetic or dermatological active principles and/or the oils, for the purpose of limiting, indeed even eliminating, their transfer on to a substrate with which the film is brought into contact. The amount of polymer depends on the amount of coloring materials and/or active principles and/or oils present in the composition. In practice, the amount of polymer can be greater than 2% by weight (as active material) with respect to the total weight of the composition.

Another subject of the invention is the use, in a cosmetic or hygiene composition for topical application or for the manufacture of a dermatological or pharmaceutical composition for topical application, of particles of at least one polymer which are dispersed in a liquid fatty phase and which are stabilized at the surface by a stabilizing agent, the fatty phase being thickened by a fat-soluble rheological agent resulting from the polymerization of at least one monomer possessing an ethylenic bond, to decrease, indeed even eliminate, the transfer of the composition film deposited on human lips and/or skin to a substrate brought into contact with the film and/or lips and to retain its gloss.

A further subject of the invention is a process for the cosmetic care of or for making up the lips, superficial body growths or skin, which comprises applying to the lips, superficial body growths or skin respectively, a cosmetic composition as defined above.

A further subject of the invention is a process for limiting, indeed even eliminating, the transfer of a composition for making up or caring for the skin or lips onto a substrate other than the skin and the lips, comprising a liquid fatty phase and at least one ingredient chosen from coloring materials and cosmetic, dermatological, hygiene and pharmaceutical active principles, which comprises introducing into the liquid fatty phase polymer particles which are dispersible in the liquid fatty phase and which can be stabilized at the surface by a stabilizing agent and at least one fat-soluble rheological agent resulting from the polymerization of at least one monomer possessing an ethylenic bond.

One advantage of the use of a dispersion of particles in a composition of the invention is that the particles remain as individual particles in the fatty phase, without forming agglomerates, which would not be the case with inorganic particles of nanometric size. Another advantage of the polymer dispersion is the possibility of obtaining very fluid compositions (on the order of 130 centipoises), even in the presence of a high amount of polymer.

Yet another advantage of such a dispersion is that it is possible to grade, as desired, the size of the polymer particles and to adjust their “polydispersity” in size during the synthesis. It is thus possible to obtain particles of very small size, which are invisible to the naked eye when in the composition and when applied to the skin or the lips. This feature would be impossible with pigments in particulate form, their composition not allowing the mean size of the particles to be varied.

In addition, it has been found that the composition according to the invention exhibits particularly advantageous qualities of spreading over and of adhesion to the skin, semi-mucous membranes or mucous membranes, as well as a smooth and pleasant touch. This composition has, in addition, the advantage of being easy to remove, particularly with a conventional make-up removal milk. This aspect is entirely remarkable, since the compositions of the prior art with high “transfer-free” properties are very difficult to remove. In general, these compositions are sold with a specific make-up removal product, which introduces an additional restriction for the user.

The composition according to the invention thus advantageously comprises one or more stable dispersions of generally spherical particles of one or more polymers in a physiologically acceptable liquid fatty phase. These dispersions can be provided in the form of nanoparticles of polymers as a stable dispersion in said fatty phase. The nanoparticles preferably have a size ranging from 5 to 600 nm, given that, beyond approximately 600 nm, the dispersions of particles become much less stable.

A further advantage of the polymer dispersion of the composition of the invention is the possibility of varying the glass transition temperature (Tg) of the polymer or of the polymeric system (polymer plus optional additive of the plasticizer type) and of thus changing from a hard polymer to a more or less soft polymer, making it possible to adjust the mechanical properties of the composition as a function of the envisaged application.

It is possible to use polymers, which can form films, preferably having a low Tg of less than or equal to the temperature of the skin, especially less than 40° C. A dispersion is thus obtained which can form a film when applied to a substrate, which is not the case when using dispersions of inorganic pigments according to the prior art.

The polymers which can be used in the composition of the invention preferably have a molecular weight on the order of 2000 to 10,000,000 and a Tg of −100° C. to 300° C., and more preferably of −10° to 50° C.

When the polymer exhibits a glass transition temperature too high for the desired application, it may be combined with a plasticizer, to lower the temperature of the mixture used. The plasticizer can be chosen from the plasticizers normally used in the field of application and from compounds which can be solvents of the polymer.

Examples of polymers which can form films include acrylic or vinyl radical homopolymers or copolymers, preferably having a Tg of less than or equal to 40° C., and more preferably ranging from −10° to 30° C.

Examples of polymers which cannot form films include optionally crosslinked, vinyl or acrylic, radical homopolymers or copolymers, preferably having a Tg of greater than or equal to 40° C., and more preferably ranging from 45° to 150° C.

The term “radical polymer” is understood to mean a polymer obtained by polymerization of monomers possessing unsaturation, in particular, ethylenic unsaturation, each monomer being capable of homopolymerizing (unlike polycondensates). The radical polymers can be vinyl polymers or copolymers, for example acrylic polymers.

The vinyl polymers can result from the polymerization of monomers possessing ethylenic unsaturation having at least one acid group and/or esters of these acid monomers and/or amides of these acids.

Use may be made, as monomers carrying acid groups, of α,β-ethylenically unsaturated carboxylic acids, such as acrylic acid, methacrylic acid, crotonic acid, maleic acid or itaconic acid. Use is preferably made of (meth)acrylic acid and crotonic acid, and more preferably (meth)acrylic acid.

The esters of acid monomers are advantageously chosen from esters of (meth)acrylic acid (also known as (meth)acrylates), such as alkyl (meth)acrylates, in particular, C₁-C₂₀ alkyl (meth)acrylates, preferably C₁-C₈ alkyl (meth)acrylates, aryl (meth)acrylates, especially C₆-C₁₀ aryl (meth)acrylates, or hydroxyalkyl (meth)acrylates, especially C₂-C₆ hydroxyalkyl (meth)acrylates. Examples of alkyl (meth)acrylates include methyl, ethyl, butyl, isobutyl, 2-ethylhexyl and lauryl (meth)acrylate. Examples of hydroxyalkyl (meth)acrylates include hydroxyethyl (meth)acrylate or 2-hydroxypropyl (meth)acrylate. Examples of aryl (meth)acrylates include benzyl or phenyl acrylate.

The particularly preferred esters of (meth)acrylic acid are alkyl (meth)acrylates.

Use is preferably made, as radical polymers, of copolymers of (meth)acrylic acid and of alkyl (meth)acrylate, especially C₁-C₄ alkyl (meth)acrylate. Use may more preferably be made of methyl acrylates, optionally copolymerized with acrylic acid.

Examples of amides of the acid monomers include (meth)acrylamides and, in particular, N-alkyl(meth)acrylamides, especially N—(C₂-C₁₂ alkyl)(meth)acrylamides, such as N-ethylacrylamide, N-(t-butyl)acrylamide or N-octylacrylamide, or N,N-di(C₁-C₄ alkyl)(meth)acrylamides.

The vinyl polymers can also result from the polymerization of monomers possessing ethylenic unsaturation having at least one amine group in the free or else partially or completely neutralized form or, alternatively, in the partially or completely quaternized form. Such monomers can be, for example, dimethylaminoethyl (meth)acrylate, (dimethylaminoethyl)methacrylamide, vinylamine, vinylpyridine or diallyldimethylammonium chloride.

The vinyl polymers can also result from the homopolymerization or from the copolymerization of at least one monomer chosen from vinyl esters and styrene monomers. In particular, these monomers can be polymerized with acid monomers and/or their esters and/or their amides, such as those mentioned above. Examples of vinyl esters include vinyl acetate, vinyl propionate, vinyl neodecanoate, vinyl pivalate, vinyl benzoate and vinyl t-butylbenzoate. Examples of styrene monomers include styrene and α-methylstyrene.

The list of the monomers given is not limiting and it is possible to use any monomer known to a person skilled in the art coming within the categories of acrylic and vinyl monomers (including monomers modified by a silicone chain).

Examples of other vinyl monomers include:

N-vinylpyrrolidone, vinylcaprolactam, vinyl-N—(C₁-C₆ alkyl)pyrroles, vinyloxazoles, vinylthiazoles, vinylpyrimidines and vinylimidazoles,

olefins, such as ethylene, propylene, butylene, isoprene and butadiene.

The vinyl polymer can be crosslinked using a difunctional monomer, in particular, comprising at least two ethylenic unsaturations, such as ethyl glycol dimethacrylate or diallyl phthalate.

The polymers of the invention can be chosen, without implied limitation, from the following polymers or copolymers: polyurethanes, polyurethane-acrylics, polyureas, polyurea-polyurethanes, polyester-polyurethanes, polyether-polyurethanes, polyesters, polyesteramides, polyesters with a fatty chain or alkyds; acrylic and/or vinyl polymers or copolymers; acrylic-silicone copolymers; polyacrylamides, silicone polymers such as silicone polyurethanes or acrylics, fluorinated polymers, and their mixtures.

The liquid fatty phase of the composition can be composed of any cosmetically or dermatologically acceptable oil and generally physiologically acceptable oil chosen, in particular, from carbon-comprising, hydrocarbon-comprising, fluorinated and/or silicone oils of mineral, animal, plant or synthetic origin, alone or as a mixture insofar as they form a homogeneous and stable mixture and insofar as they are compatible with the use envisaged.

The term “liquid fatty phase” is understood to mean any non-aqueous medium which is liquid at room temperature (25° C.) and atmospheric pressure. This fatty phase can comprise a volatile liquid fatty phase and/or a non-volatile fatty phase.

The term “volatile fatty phase” is understood to mean any non-aqueous medium capable of evaporating from the skin or lips in less than one hour. This volatile phase, in particular, comprises oils having a vapor pressure, at room temperature and atmospheric pressure, ranging from 10⁻³ to 300 mm of Hg (0.13 Pa to 40,000 Pa).

The complete liquid fatty phase of the composition can represent from 5% to 97.90% of the total weight of the composition, and preferably represents from 20 to 85%. The non-volatile part can represent from 0 to 80% of the total weight of the composition, and better still represents from 1 to 50%.

Examples of liquid fatty phases which can be used in the invention include hydrocarbon-comprising oils, such as liquid paraffin or liquid petrolatum, mink oil, turtle oil, soybean oil, perhydrosqualene, sweet almond oil, calophyllum oil, palm oil, grape seed oil, sesame oil, maize oil, parleam oil, arara oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters, such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, di(2-ethylhexyl) succinate, diisostearyl malate, glyceryl triisostearate or diglyceryl triisostearate; higher fatty acids, such as myristic acid, palmitic acid, stearic acid, behenic acid, oleic acid, linoleic acid, linolenic acid or isostearic acid; higher fatty alcohols, such as cetanol, stearyl alcohol or oleyl alcohol, linoleyl or linolenyl alcohol, isostearyl alcohol or octyldodecanol; silicone oils, such as polydimethylsiloxanes (PDMS), which are optionally phenylated, such as phenyltrimethicones, or which are optionally substituted by optionally fluorinated aliphatic and/or aromatic groups or by functional groups, such as hydroxyl, thiol and/or amine groups; polysiloxanes modified by fatty acids, fatty alcohols or polyoxyalkylenes, fluorinated silicones or perfluorinated oils.

Use may advantageously be made of one or more oils which are volatile at room temperature. These volatile oils are favorable in the production of a film with complete “transfer-free” properties. After evaporation of these oils, a supple and non-sticky film-forming deposit is obtained on the skin or mucous membranes which respectively follows the movements of the skin or lips on which the composition is applied. In addition, these volatile oils facilitate the application of the composition to the skin, mucous membranes and superficial body growths. They can be hydrocarbon-comprising or silicone oils optionally comprising alkyl or alkoxy groups which are pendent or at the end of the silicone chain.

Examples of volatile oil which can be used in the invention include linear or cyclic silicones having from 2 to 7 silicon atoms, these silicones optionally comprising alkyl or alkoxy groups having from 1 to 10 carbon atoms, and C₈-C₁₆ isoparaffins. These volatile oils represent, in particular, from 20 to 97.90% of the total weight of the composition, and better still represent from 30 to 75%.

Examples of volatile oils which can be used in the invention include octamethylcyclotetrasiloxane, decamethylcyclopentasiloxane, hexadecamethylcyclohexasiloxane, heptamethylhexyltrisiloxane, heptamethyloctyltrisiloxane or C₈-C₁₆ isoparaffins, such as “Isopars,” Permetyls and, in particular, isododecane.

In a specific embodiment of the invention, the liquid fatty phase is chosen from the group comprising:

non-aqueous liquid compounds having an overall solubility parameter according to the Hansen solubility space of less than 17 (MPa)^{1/2},

or monoalcohols having an overall solubility parameter according to the Hansen solubility space of less than or equal to 20 (MPa)^{1/2},

or their mixtures.

The overall solubility parameter δ according to the Hansen solubility space is defined in the article “Solubility Parameter Values” by Eric A. Grulke in the work “Polymer Handbook,” 3rd edition, Chapter VII, pages 519-559, by the relationship:

δ=(d_{D}²+d_{P}²+d_{H}²)^{1/2}, in which:

d_{D} characterizes the London dispersion forces resulting from the formation of dipoles induced during molecular impacts,

d_{P} characterizes the forces of Debye interactions between permanent dipoles,

d_{H} characterizes the forces of specific interactions (hydrogen bond, acid/base or donor/acceptor type and the like). The definition of the solvents in the three-dimensional solubility space according to Hansen is given in the article by C. M. Hansen: “The three-dimensional solubility parameters,” J. Paint Technol., 39, 105(1967).

Examples of liquid fatty phases having an overall solubility parameter according to the Hansen solubility space of less than or equal to 17 (MPa)^{1/2} include vegetable oils formed by esters of fatty acids and polyols, in particular, triglycerides, such as sunflow r oil, sesame oil or rapeseed oil, or esters derived from long-chain acids or alcohols, i.e., having from 6 to 20 carbon atoms, in particular, esters of formula RCOOR′ in which R represents the residue of a higher fatty acid comprising from 7 to 19 carbon atoms and R′ represents a hydrocarbon-atom-comprising chain comprising from 3 to 20 carbon atoms, such as palmitates, adipates and benzoates, in particular, diisopropyl adipate. Examples of hydrocarbons and, in particular, of liquid paraffin include liquid petrolatum or hydrogenated polyisobutylene, isododecane, or, alternatively, “Isopars,” volatile isoparaffins. Other examples include silicone oils, such as polydimethylsiloxanes and polymethylphenylsiloxanes, optionally substituted by optionally fluorinated aliphatic and/or aromatic groups or by functional groups, such as hydroxyl, thiol and/or amine groups, and volatile silicone oils, in particular, cyclic oils. Mention may also be made of solvents, alone or as a mixture, chosen from (i) linear, branched or cyclic esters having more than 6 carbon atoms, (ii) ethers having more than 6 carbon atoms, or (iii) ketones having more than 6 carbon atoms. Monoalcohols having an overall solubility parameter according to the Hansen solubility space of less than or equal to 20 (MPa)^{1/2} is understood to mean aliphatic fatty alcohols having at least 6 carbon atoms, the hydrocarbon-atom-comprising chain not comprising a substitution group. Examples of monoalcohols according to the invention include oleyl alcohol, decanol, dodecanol, octadecanol and linoleyl alcohol.

Use may also be made, as non-aqueous medium, of those disclosed in French Patent Application FR-A-2,710,646 of L.V.M.H.

The choice of the non-aqueous medium is made by a person skilled in the art according to the nature of the monomers constituting the polymer and/or the nature of the stabilizing agent, as indicated below.

The polymer dispersion can be manufactured as disclosed in European Patent Application EP-A-749,747. The polymerization can be carried out as a dispersion, i.e., by precipitation of the polymer during formation, with protection of the particles formed with a stabilizing agent.

A mixture comprising the starting monomers and a radical initiator is thus prepared. This mixture is dissolved in a solvent known, in the continuation of the present description, as the “synthesis solvent.” When the fatty phase is a non-volatile oil, the polymerization can be carried out in an apolar organic solvent (synthesis solvent), the non-volatile oil (which must be miscible with the synthesis solvent) can then be added and the synthesis solvent can be selectively distilled.

A synthesis solvent is thus chosen such that the starting monomers and the radical initiator are soluble therein and the polymer particles obtained are insoluble therein, in order for them to precipitate therefrom as they are formed. In particular, the synthesis solvent can be chosen from alkanes, such as heptane, isododecan or cyclohexane.

When the fatty phase chosen is a volatile oil, the polymerization can be carried out directly in the oil, which thus also acts as the synthesis solvent. The monomers must also be soluble therein, as well as the radical initiator, and the polymer obtained must be insoluble therein.

The monomers are preferably present in the synthesis solvent, before polymerization, in a proportion of from 5 to 20% by weight of the reaction mixture. All the monomers can be present in the solvent before the beginning of the reaction or a portion of the monomers can be added as the polymerization reaction proceeds.

The radical initiator can be azobisisobutyronitrile or tert-butyl peroxy(2-ethylhexanoate).

Stabilizing Agent

The polymer particles are stabilized at the surface, as the polymerization proceeds, by virtue of a stabilizing agent which can be a sequential polymer, a grafted polymer and/or a random polymer, alone or as a mixture. The stabilization can be carried out by any known means and, in particular, by direct addition of the sequential polymer, grafted polymer and/or random polymer during the polymerization.

The stabilizing agent is preferably also present in the mixture before polymerization. However, it is possible to add it continuously, in particular, when the monomers are also added continuously.

Use can be made of from 2 to 30% by weight of stabilizing agent with respect to the starting mixture of monomers, and preferably of from 5 to 20% by weight.

When a grafted and/or sequential polymer is used as stabilizing agent, the synthesis solvent is chosen such that at least a portion of the grafts or sequences of the stabilizing polymer is soluble in the solvent, the other portion of the grafts or sequences not being soluble therein. The stabilizing polymer used during the polymerization must be soluble, or dispersible, in the synthesis solvent. Furthermore, the choice is preferably made of a stabilizing agent for which the insoluble sequences or grafts exhibit a degree of affinity for the polymer formed during the polymerization.

Examples of grafted polymers include silicone polymers grafted with a hydrocarbon-comprising chain or hydrocarbon-comprising polymers grafted with a silicone chain.

Grafted copolymers having, for example, an insoluble backbone of polyacrylic type with soluble grafts of poly(12-hydroxystearic acid) type are also suitable.

Thus, use may be made of grafted or sequential block copolymers comprising at least one block of polyorganosiloxane type and at least one block of a radical polymer, such as grafted copolymers of acrylic/silicone type, which can be employed when the non-aqueous medium is a silicone medium.

Use may also be made of grafted or sequential block copolymers comprising at least one block of polyorganosiloxane type and at least one block of a polyether. The polyorganosiloxane block can be a polydimethylsiloxane or a poly(C₂-C₁₈)alkylmethylsiloxane; the polyether block can be a poly(C₂-C₁₈ alkylene oxide), especially polyoxyethylene and/or polyoxypropylene. Use may be made of dimethicone copolyols or (C₂-C₁₈)alkyl dimethicone copolyols, such as those sold under the name “Dow Corning 3225C” by the company Dow Corning, or lauryl methicones, such as those sold under the name “Dow Corning Q2-5200” by the company Dow Corning.

Examples of grafted or sequential block copolymers include those comprising at least one block resulting from the polymerization of at least one ethylenic monomer possessing one or more ethylenic bonds which are optionally conjugated, such as ethylene or dienes, such as butadiene and isoprene, and at least one block of a vinyl polymer, and better still, a styrene polymer. When the ethylenic monomer comprises several ethylenic bonds which are optionally conjugated, the residual ethylenic unsaturations after the polymerization are generally hydrogenated. Thus, in a known way, the polymerization of isoprene results, after hydrogenation, in the formation of an ethylene-propylene block and the polymerization of butadiene results, after hydrogenation, in the formation of an ethylene-butylene block. Examples of these polymers include sequential copolymers, in particular, of “diblock” or “triblock” type, the polystyrene/polyisoprene or polystyrene/polybutadiene type, such as those sold under the name of “Luvitol HSB” by BASF, the polystyrene/copoly(ethylene-propylene) type, such as those sold under the name of “Kraton” by Shell Chemical Co., or the polystyrene/copoly(ethylene-butylene) type (see hereinbelow the commercial names of the rheological agent). These polymers are generally known as copolymers of hydrogenated or non-hydrogenated diene.

Examples of grafted or sequential block copolymers comprising at least one block resulting from the polymerization of at least one ethylenic monomer possessing one or more ethylenic bonds and at least one block of an acrylic polymer include poly(methyl methacrylate)/polyisobutylene bi- or trisequential copolymers or of grafted copolymers with a poly(methyl methacrylate) backbone and with polyisobutylene grafts.

Examples of grafted or sequential block copolymers comprising at least one block resulting from the polymerization of at least one ethylenic monomer possessing one or more ethylenic bonds and at least one block of a polyether, such as a poly(C₂-C₁₈ alkylene oxide), in particular, polyoxyethylenated and/or polyoxypropylenated, include polyoxyethylene/polybutadiene or polyoxyethylene/polyisobutylene bi- or trisequential copolymers.

When a random polymer is used as stabilizing agent, it is chosen so that it has a sufficient amount of groups rendering it soluble in the envisaged synthesis solvent.

It is thus possible to employ copolymers based on alkyl acrylates or methacrylates resulting from C₁-C₄ alcohols and on alkyl acrylates or methacrylates resulting from C₈-C₃₀ alcohols, for example, the stearyl methacrylate/methyl methacrylate copolymer.

When the synthesis solvent is apolar, it is preferable to choose, as the stabilizing agent, a polymer which introduces the most complete possible covering of the particles, several chains of stabilizing polymers then being adsorbed on one particle of polymer obtained by polymerization.

In this case, it is then preferable to use, as the stabilizing agent, either a grafted polymer or a sequential polymer, so as to have a better interfacial activity. This choice is preferred because the sequences or grafts which are insoluble in the synthesis solvent contribute a bulkier covering to the surface of the particles.

When the liquid synthesis solvent comprises at least one silicone oil, the stabilizing agent is preferably chosen from grafted or sequential block copolymers comprising at least one block of polyorganosiloxane type and at least one block of a radical polymer or of a polyether or of a polyester, such as polyoxypropylenated and/or polyoxyethylenated blocks.

When the liquid fatty phase does not comprise a silicone oil, the stabilizing agent is preferably chosen from:

(a) grafted or sequential block copolymers comprising at least one block of polyorganosiloxane type and at least one block of a radical polymer or of a polyether or of a polyester,

(b) copolymers of alkyl acrylates or methacrylates resulting from C₁-C₄ alcohols and of alkyl acrylates or methacrylates resulting from C₈-C₃₀ alcohols,

(c) grafted or sequential block copolymers comprising at least one block resulting from the polymerization of at least one ethylenic monomer possessing conjugated ethylenic bonds,

and at least one block of a vinyl or acrylic polymer or of a polyether or of a polyester, or their mixtures.

Use is preferably made of diblock polymers as the stabilizing agent.

Rheological Agent

According to the invention, it is possible to use one or more fat-soluble rheological agents in the composition of the invention. This or these rheological agents are fat-soluble agents capable of thickening and/or gelling the composition. They are present in an amount which is effective in increasing the viscosity of the composition until a gel is obtained, namely a product which does not flow under its own weight, even a stick. The amount of rheological agent depends on the viscosity desired for the final composition. In practice, the rheological agent or the mixture of rheological agents represents from 0.05 to 20% of the total weight of the composition, preferably from 0.1% to 10% by weight, and better still from 0.5 to 5%.

The rheological agents according to the invention each result from the polymerization or copolymerization of an ethylenic monomer comprising one or more ethylenic bonds which are preferably conjugated (or diene). This or these agents are, in particular, vinyl, acrylic or methacrylic copolymers which can be sequential and, in particular, of diblock or triblock type, indeed even of multiblock type or star type.

The ethylenic rheological agent or agents according to the invention preferably comprise a styrene (S) block, an alkylstyrene (AS) block, an ethylene/butylene (EB) block, an ethylene/propylene (EP) block, a butadiene (B) block, an isoprene (I) block, an acrylate (A) block, a methacrylate (MA) block or a combination of these blocks.

In particular, use is made, as rheological agent, of a copolymer comprising at least one styrene block. Use is preferably made of a triblock copolymer and, in particular, those of the polystyrene/polyisoprene or polystyrene/polybutadiene type, such as those sold under the name of “Luvitol HSB” by BASF, and those of the polystyrene/copoly(ethylene/propylene) type or of the polystyrene/copoly(ethylene-butylene) type, such as those sold under the trade name “Kraton” by Shell Chemical Co., or Gelled Permethyl 99A by Penreco. Use may also be made of styrene-methacrylate copolymers.

Examples of rheological agents which can be used in the composition of the invention include, for example, Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton G1750X (EP) multibranched, Kraton G1765X (EP) multibranched, Kraton D-1101 (SBS), Kraton D-1102 (SBS), Kraton D-1107 (SiS), Gelled Permethyl 99A-750, Gelled Permethyl 99A-753-58 (mixture of star block polymer and of triblock), Gelled Permethyl 99A-753-59 (mixture of star block polymer and of triblock), Versagel 5970 and Versagel 5960 from Penreco (mixture of star polymer and of triblock in isododecane), OS 129880, OS 129881 and OS 84383 from Lubrizol (styrene-methacrylate copolymer).

This or these fat-soluble rheological agents make possible a thickening of the composition while retaining a glossy appearance with regard to the composition as well as with regard to the film deposited on the lips and/or body. This result clearly emerges from Table I below.

Additives

The dispersions of gelled polymers obtained according to the invention can then be used in a composition, in particular, a cosmetic, dermatological, pharmaceutical and/or hygiene composition, such as a composition for caring for or for making up the skin or lips, or, alternatively, a hair composition or an anti-sun composition or a composition for coloring or artificially tanning the skin.

Depending on the application, it is possible to choose to use dispersions of polymers, which can or cannot form films, in volatile or non-volatile oils.

The composition of the invention can advantageously comprise one or more coloring materials comprising one or more pulverulent compounds and/or one or more fat-soluble or water-soluble colorants, for example in a proportion of from 0 to 70% of the total weight of the composition and, in particular, of from 0.01 to 70%. The pulverulent compounds can be chosen from pigments and/or pearlescent agents and/or fillers commonly used in cosmetic or dermatological compositions. The pulverulent compounds advantageously represent from 0 to 50% and, in particular, from 0.1 to 50%, of the total weight of the composition, and better still from 1 to 40%. The smaller the amount of pulverulent compounds, the better the transfer-free and comfort qualities. The fact that the transfer-free properties are enhanced as the amount of pulverulent compounds decreases is entirely surprising, because, until now, the transfer-free properties of the compositions of the prior art were enhanced with the amount of pulverulent compounds. Conversely, their discomfort, their gloss and their dryness on the skin or mucous membranes increased.

Furthermore, the transfer-free property is enhanced with the amount of polymer which is dispersible in the liquid fatty phase. In practice, the polymer can represent, as active material, up to 60% (as active material or dry matter) of the total weight of the composition. On using more than 12% by weight of active polymer material and of non-volatile oil in the composition and up to 60%, a total transfer-free film is obtained. Between 2% and 12%, the transfer-free effect is significant without, however, being complete. The transfer-free properties can thus be adjusted as desired, which was impossible with the transfer-free compositions of the prior art, without harming the comfort of the film deposited.

The pigments can be white or colored, inorganic and/or organic, interferential or non-interferential. Examples of inorganic pigments include titanium dioxide, optionally treated at the surface, or zirconium or cerium oxides, as well as iron or chromium oxides, manganese violet, ultramarine blue, chromium hydrate and ferric blue. Examples of organic pigments include carbon black, pigments of D & C type and lakes based on cochineal carmine or on barium, strontium, calcium or aluminium.

The pearlescent pigments can be chosen from white pearlescent pigments, such as mica covered with titanium oxide or with bismuth oxychloride, colored pearlescent pigments, such as titanium oxide-coated mica with iron oxides, titanium oxide-coated mica with, in particular, ferric blue or chromium oxide, or titanium oxide-coated mica with an organic pigment of the abovementioned type, as well as pearlescent pigments based on bismuth oxychloride.

The fillers can be inorganic or organic, lamellar or spherical. Examples include talc, mica, silica, kaolin, nylon (Orgasol from Atochem), poly-β-alanine and polyethylene powders, Teflon, lauroyllysine, starch, boron nitride, tetrafluoroethylene polymer powders, hollow microspheres, such as Expancel (Nobel Industrie), polytrap (Dow Corning) and silicone resin microbeads (Tospearls from Toshiba, for example), precipitated calcium carbonate, magnesium carbonate and carbonate hydroxide, hydroxyapatite, hollow silica microspheres (Silica Beads from Maprecos), glass or ceramic microcapsules, or metal soaps derived from organic carboxylic acids having from 8 to 22 carbon atoms, preferably from 12 to 18 carbon atoms, for example zinc, magnesium or lithium stearate, zinc laurate or magnesium myristate.

The fat-soluble colorants are, for example, Sudan red, DC Red 17, DC Green 6, β-carotene, soybean oil, Sudan brown, DC Yellow 11, DC Violet 2, DC Orange 5 or Quinoline yellow. They can represent from 0 to 20% and preferably 0.01 to 20% of the weight of the composition, and better still from 0.1 to 6%. The water-soluble colorants are, for example, beetroot juice or methylene blue and can represent up to 6% of the total weight of the composition.

The composition of the invention can additionally comprise one or more cosmetic or dermatological active principles, such as those conventionally used.

Examples of cosmetic, dermatological, hygiene or pharmaceutical active principles which can be used in the composition of the invention include moisturizing agents, vitamins, essential fatty acids, sphingolipids or sunscreen agents. These active principles are used in an amount usual to a person skilled in the art and preferably at concentrations of from 0 to 20% and, in particular, of from 0.001 to 20% of the total weight of the composition.

The polymer of the composition of the invention makes possible the formation of a film on the skin, lips and/or mucous membranes which forms a network which traps the coloring materials (including the fillers) and/or the active principles. Depending on the relative amount of coloring materials used, or used with respect to the amount of stabilized polymer, it is possible to obtain a more or less glossy and more or less transfer-free film.

The composition according to the invention can additionally comprise, according to the type of application envisaged, the constituents conventionally used in the fields under consideration, which are present in an amount appropriate to the desired pharmaceutical dosage form.

In particular, it can comprise, in addition to the liquid fatty phase in which the polymer is stabilized, additional fatty phases which can be chosen from waxes, oils, gums and/or pasty fatty substances of plant, animal, mineral or synthetic origin, indeed silicone origin, and their mixtures.

Examples of waxes which are solid at room temperature which can be present in the composition according to the invention include hydrocarbon-comprising waxes, such as beeswax, carnauba wax, candelilla wax, ouricury wax, japan wax, cork fibre or sugarcane waxes, paraffin or lignite waxes, microcrystalline waxes, lanolin wax, montan wax, ozokerites, polyethylene waxes, waxes obtained by Fischer-Tropsch synthesis, hydrogenated oils, fatty esters and glycerides which are solid at 25° C. It is also possible to use silicone waxes, for example, alkyl, alkoxy and/or esters of polymethylsiloxane. The waxes can be provided in the form of stable dispersions of colloidal wax particles, such that they can be prepared according to known methods, for example of “Microemulsions, Theory and Practice,” edited by L. M. Prince, Academic Press (1977), pages 21-32. An example of a wax which is liquid at room temperature is jojoba oil.

The waxes can be present in a proportion of from 0 to 50% by weight in the composition and better still of from 5 to 20%, for the purpose of not excessively decreasing the gloss of the composition and of the film deposited on the lips and/or skin.

The composition can additionally comprise any additive conventionally used in such compositions, such as thickeners, other than the ethylenic rheological agent, antioxidants, fragrances, preservatives, surfactants, or fat-soluble polymers, such as polyalkylenes, in particular, polybutene, polyacrylates and silicone polymers which are compatible with the fatty phase, and polyvinylpyrrolidone derivatives. Of course, a person skilled in the art will take care to choose this or these possible additional compounds, and/or their amount, so that the advantageous properties of the composition according to the invention are not, or not substantially, detrimentally affected by the envisaged addition.

In a specific embodiment of the invention, the compositions according to the invention can be prepared conventionally by a person skilled in the art. They can be provided in the form of a cast product and, for example, in the form of a stick or in the form of a disk which can be used by direct contact or with a sponge. In particular, they find an application as cast foundation, cast blusher or eyeshadow, lipstick, base or balm for caring for the lips, or product for concealing rings under the eyes. They can also be provided in the form of a supple paste, with a dynamic viscosity at 25° C. of the order of 1 to 40 Pa.s, or, alternatively, of a more or less fluid cream or gel. They can then constitute foundations or lipsticks, anti-sun products or products for coloring the skin.

The compositions of the invention are advantageously anhydrous and can comprise less than 5% of water with respect to the total weight of the composition. They can then be provided in the form of an oily gel, of an oily liquid or oil, of a paste or of a stick or, alternatively, in the form of a vesicular dispersion comprising ionic and/or non-ionic lipids. They can also be provided in the form of a simple or multiple emulsion with an oily or aqueous continuous phase or of an oily dispersion in an aqueous phase by virtue of vesicules comprising ionic and/or non-ionic lipids. These pharmaceutical dosage forms are prepared according to the methods usual in the fields under consideration.

These compositions for topical application can constitute, in particular, a cosmetic, dermatological, hygiene or pharmaceutical composition for protecting, treating or caring for the face, neck, hands or body (for example, care cream, anti-sun oil or body gel), a make-up composition (for example, make-up gel, cream or stick) or a composition for the artificial tanning or protection of the skin.

The present invention is further illustrated by the following examples which are designed to teach those of ordinary skill in the art how to practice the invention. The following examples are merely illustrative of the invention and should not be construed as limiting the invention as claimed. The percentages are percentages by weight.

### Example 1 of Polymer Dispersion

A dispersion of polymethyl methacrylate crosslinked with ethylene glycol dimethacrylate was prepared in isododecane according to the method of Example 2 of European Patent Application EP-A-749,746; Isopar L being replaced by isododecane. A dispersion of polymethyl methacrylate particles stabilized at the surface in isododecane by a polystyrene/copoly(ethylene-propylene) sequential diblock copolymer sold under the name of Kraton G1701 (Shell) was thus obtained, the dispersion having a dry matter content of 19.7% by weight and a mean size of the particles of 135 nm (polydispersity: 0.05) and a Tg of 100° C. This copolymer cannot form a film at room temperature.

### Example 2 of Polymer Dispersion

A dispersion of non-crosslinked copolymer of methyl acrylate and of acrylic acid, in a 95/5 ratio, was prepared in isododecane, according to the method of Example 1 of European Patent Application EP-A-749,746; heptane being replaced by isododecane. A dispersion of poly(methyl acrylate/acrylic acid) particles stabilized at the surface in isododecane by a polystyrene/copoly-(ethylene-propylene) sequential diblock copolymer sold under the name of Kraton G1701 (Shell) was thus obtained, the dispersion having a dry matter content of 19% by weight and a mean size of the particles of 165 nm (polydispersity: 0.05) and a Tg of 13° C. This copolymer can form a film.

### Example 3 of Polymer Dispersion

A dispersion of non-crosslinked copolymer of methyl acrylate and of acrylic acid, in a 95/5 ratio, was prepared in isododecane, according to the method of Example 1 of European Patent Application EP-A-749,746; heptane being replaced by isododecane. A dispersion of poly(methyl acrylate/acrylic acid) particles stabilized at the surface in isododecane by a polystyrene/copoly-(ethylene-propylene) sequential diblock copolymer sold under the name of Kraton G1701 (Shell) was thus obtained, which dispersion has a dry matter content of 22.25% by weight and a mean size of the particles of 180 nm (polydispersity: 0.05) and a Tg of 20° C. This copolymer can form a film.

Comparative Tests

Pigments and a gelling agent according to the invention and according to the prior art were introduced into the dispersions of Examples 2 and 3. The viscosity of the compositions obtained was subsequently measured at 25° C. using a viscometer of Brookfield LV type rotating at 100 rev/min and equipped with an LV4 or LV3 rotor, depending on the viscosity. The gloss was also measured using a glossmeter of Byk Gardner type.

The results are given in Table I below:

TABLE I

| | Kraton | Kraton | Carnauba | | KSG |
|---|---|---|---|---|---|
| | diblock | diblock | wax | Bentone 34 | X-21-5432 |
| % Dispersion, Example 2 | 87.4 | | 88.7 | 85.47 | 84.14 |
| % Dispersion, Example 3 | | 92.2 | | | |
| % Gelling agent | 1.3 | 0.75 | 2 | 3.92 | 1.5 |
| % Pigments | 3 | 3 | 3 | 3 | 4 |
| Viscosity (cPs) | 1626 | 1758 | 1266 | 3210 | 285.6 |
| | LV4 rotor | LV4 rotor | LV4 rotor | LV4 rotor | LV3 rotor |
| Gloss | 77.2 | 67.1 | 11.4 | 4.7 | 2.7 |

It clearly emerges from this table that the gelling agents of the composition according to the invention provide the compositions with a high gloss, which is not the case with the conventional gelling agents, such as waxes, Bentone 34 from Rheox, or KSG X-21-5432, sold. by the company Shin Etsu, which is a crosslinked organopolysiloxane with an active material content of 27% in polymethyltrifluoropropyl dimethylsiloxane. The composition not comprising gelling agent or pigments had a gloss of 84%.

### Example 4

Eyeliner

An eyeliner was prepared in the form of a fluid having the following composition:

| | |
|---|---|
| Hydrogenated polyisobutene | 0.77% |
| Black iron oxides | 15.00% |
| Polymer dispersion of Example 3 | 81.40% |
| Polystyrene/poly(ethylene-propylene)* | 1.30% |
| PVP**/eicosene copolymer | 0.44% |
| Phenyltrimethicone | 0.76% |
| Octyldodecanol | 0.33% |

*Kraton - G1701 (Shell Chimie)
**PVP - polyvinylpyrrolidone

This eyeliner was obtained according to the following procedure: The Kraton was dissolved in the hot polymer dispersion (approximately 50° C.) for 2 hours and then the pigments,.premilled in the oils, were added.

This eyeliner was tested by people who were experts in cosmetics. The results of the test are given hereinbelow:

application was easy and smooth,

coverage was good,

the result of the make-up was satisfactory; the glint was satiny or satiny-glossy; the line was sharp at the edge, and

adhesion was good.

The comfort was good for ⅔ of the female testers, during application and after drying. The hold was considered as good by all the women. The hold was considered as good, indeed even very good for some female testers.

### Example 5

Lip Gloss

A lip gloss was prepared in the form of a fluid having the following composition:

| | |
|---|---|
| Hydrogenated polyisobutene | 2.23% |
| PVP/eicosene copolymer | 1.28% |
| Aluminium lake of DC Red 27 | 2.00% |
| Calcium lake of DC Red 7 | 3.69% |
| DC Red 36 | 0.95% |
| Black iron oxide | 0.07% |
| Red iron oxide | 2.09% |
| Dispersion of Example 3 | 83.50% |
| Kraton G-1650 V (Shell Chimie)* | 1.00% |
| Phenyltrimethicone | 2.23% |
| Octyldodecanol | 0.96% |

*polystyrene/poly(ethylene-butylene)/polystyrene

This lip laquer was very glossy, completely transfer-free, and non-migrating. It was obtained as in Example 4.

### Example 6

Transfer-free Lipstick

A lipstick was prepared in the form of a fluid having the following composition:

| | |
|---|---|
| Hydrogenated polyisobutene | 0.77% |
| Iron oxides | 4.00% |
| Dispersion of Example 2 | 92.20% |
| Kraton G1701 (Shell Chimie) | 1.50% |
| PVP/eicosene copolymer | 0.44% |
| Phenyltrimethicone | 0.76% |
| Octyldodecanol | 0.33% |

This lipstick was prepared as in Example 4. It was subsequently tested by experts using an applicator with a foam nozzle. It was provided in a form which was fluid, glossy, soft on application, transfer-free and non-migrating.

Application was easy and fast. The charge through the foam nozzle was good and the texture was fluid and light. The applicator glided very well and the line was drawn in a single stroke. Make-up of the lips was fast; this lipstick deposited a homogeneous film which did not need to be gone over again.

Comfort was good on application for four-fifths of the female testers.

The contour was sharp on unwrinkled lips. This lipstick left a beautiful, satiny glint and good coverage.

Transfer was slight. Deposition was considered to be “traces” for 2/5 tests and “non-existent” for 3/5 tests. It was assessed by a “kiss” on a sheet of paper 2 minutes after drying.

### Example 7

Lip Gloss

A lip gloss was prepared in the form of a fluid having the following composition:

| | |
|---|---|
| Apricot kernel oil | 5.00% |
| Hydrogenated polyisobutene | 0.77% |
| Aluminium lake of DC Red 27 | 0.91% |
| Calcium lake of DC Red 7 | 1.68% |
| DC Red 36 | 0.43% |
| Black iron oxide | 0.03% |
| Red iron oxide | 0.95% |
| PVP/eicosene copolymer | 0.43% |
| Kraton G1701 (Shell Chimie) | 1.30% |
| Dispersion of Example 3 | 87.40% |
| Phenyltrimethicone | 0.77% |
| Octyldodecanol | 0.33% |

This lip gloss was prepared as in Example 4, with addition of the apricot oil to the polymer dispersion before the addition of the pigments.

### Example 8

Fluid Foundation

A foundation was prepared in the form of a fluid having the following composition:

| | |
|---|---|
| Hydrogenated polyisobutene | 7.00% |
| Yellow iron oxid | 1.17% |
| Black iron oxide | 0.26% |
| Brown iron oxide | 0.64% |
| Titanium dioxide | 7.93% |
| Polymer dispersion of Example 2 | 55.00% |
| Polystyrene/poly(ethylene-propylene) | 2.00% |
| Nylon-12 | 12.00% |
| PVP/eicosene copolymer | 4.00% |
| Phenyltrimethicone | 7.00% |
| Octyldodecanol | 3.00% |

This foundation was prepared as in Example 4. It was provided in the form of a fluid, spread well, was comfortable and had very good transfer-free properties.

### Example 9

Glossy Lipstick with Improved Transfer-free Properties

A lipstick was prepared in the form of a stick having the following composition:

| | |
|---|---|
| Sesame oil | 8.06% |
| Sodium hyaluronate | 0.06% |
| Preservative | 0.06% |
| Lanolin oil | q.s. for |
| | 100% |
| Acetylated lanolin | 4.04% |
| Arara oil | 8.06% |
| Oleyl erucate | 8.06% |
| Microcrystalline wax | 6.18% |
| Musk rose oil | 1.05% |
| Titanium dioxide | 1.81% |
| FD & C Yellow No. 6 aluminium lake | 3.35% |
| Black iron oxides | 0.06% |
| Aluminium lake of DC Red 21 | 0.06% |
| Calcium lake of DC Red 7 | 2.88% |
| Octyl methoxycinnamate | 0.42% |
| Quaternium-18 hectorite (Bentone 37 V) | 0.35% |
| Dispersion of Example 2 | 31.00% |
| Kraton G1650E (Shell Chimie) | 0.60% |
| PPG-5 lanolin wax | 4.03% |
| Polyglycerolated beeswax | 2.46% |
| Octyldodecanol | 0.31% |
| Tocopheryl acetate | 0.31% |

This lipstick was provided in the form of a stick. It was tested in comparison with a conventional lipstick not comprising the polymer dispersion or the Kraton G1650E. This conventional lipstick was a very comfortable lipstick.

Both of these lipsticks were recognized as being very easy and fast to apply, as having a supple, creamy, smooth and adherent texture. Coverage was better for the lipstick of the invention and the film formed was more homogeneous than those of the conventional lipstick. The make-up was considered as satisfactory and the edges were sharp. The film was satiny. Transfer was assessed by comparing, by half-lips, by a kiss on a filter paper 2 minutes after application. Transfer was considered as less significant (light to moderate) with the lipstick of the invention for all the female testers with respect to that of the prior art (moderate to significant). The comfort after application was considered as satisfactory for both lipsticks.

The foregoing written description relates to various embodiments of the present invention. Numerous changes and modifications may be made therein without departing from the spirit and scope of the invention as defined in the following claims.

What is claimed is:

1. A composition for topical application comprising a liquid fatty phase and polymer particles, said polymer particles being dispersed in the liquid fatty phase and stabilized at the surface by a stabilizing agent, and said liquid fatty phase being thickened by a fat-soluble rheological agent resulting from the polymerization of at least one monomer possessing an ethylenic bond.

2. A composition according to claim 1, wherein said rheological agent is chosen from vinyl, acrylic and methacrylic polymers.

3. A composition according to claim 1, wherein said rheological agent is a block copolymer.

4. A composition according to claim 3, wherein said rheological agent is a copolymer comprising at least one styrene block and at least one ethylene-propylene or ethylene-butylene or methacrylate block.

5. A composition according to claim 3, wherein said rheological agent is a triblock copolymer.

6. A composition according to claim 1, wherein said rheological agent is present in an amount ranging from 0.05 to 20% of the total weight of the composition.

7. A composition according to claim 6, wherein said rheological agent is present in an amount ranging from 0.5 to 5% of the total weight of the composition.

8. A composition according to claim 1, wherein said polymer particles dispersed in the liquid fatty phase can form a film.

9. A composition according to claim 1, further comprising at least one active principle chosen from cosmetic, dermatological, hygiene and pharmaceutical active principles.

10. A composition according to claim 1, further comprising at least one coloring material.

11. A composition according to claim 1, wherein said polymer particles dispersed in the liquid fatty phase are chosen from radical polymers, polycondensates, polymers of natural origin and their mixtures.

12. A composition according to claim 11, wherein said polymer particles dispersed in the liquid fatty phase are chosen from polyurethanes; polyurethane-acrylics; polyureas; polyurea-polyurethanes; polyester-polyurethanes; polyether-polyurethanes; polyesters; polyesteramides; polyesters with a fatty chain or alkyds; acrylic and/or vinyl polymers or copolymers; acrylic-silicone copolymers; polyacrylamides; silicone polymers, fluorinated polymers; and mixtures thereof.

13. A composition according to claim 1, wherein said liquid fatty phase comprises at least one carbon-containing, hydrocarbon-containing, fluorinated or silicone oil of mineral, animal, plant or synthetic origin, alone or as a mixture.

14. A composition according to claim 13, wherein said liquid fatty phase is chosen from liquid paraffin, liquid petrolatum, mink oil, turtle oil, soybean oil, perhydrosqualene, sweet almond oil, calophyllum oil, palm oil, parleam oil, grape seed oil, sesame oil, maize oil, rapeseed oil, sunflower oil, cottonseed oil, apricot oil, castor oil, avocado oil, jojoba oil, olive oil or cereal germ oil; esters of lanolic acid, of oleic acid, of lauric acid or of stearic acid; fatty esters; higher fatty acids; higher fatty alcohols; silicone oils; polysiloxanes modified by fatty acids, fatty alcohols or polyoxyalkylenes; fluorinated silicones or perfluorinated oils; and volatile oils.

15. A composition according to claim 1, wherein said liquid fatty phase comprises at least one oil which is volatile at room temperature and atmospheric pressure.

16. A composition according to claim 1, wherein said stabilizing agent is chosen from sequential polymers, grafted polymers, random polymers and mixtures thereof.

17. A composition according to claim 16, wherein said stabilizing agent is chosen from silicone polymers grafted with a hydrocarbon-containing chain; hydrocarbon-containing polymers grafted with a silicone chain; grafted copolymers having an insoluble backbone of polyacrylic type with soluble grafts of poly(12-hydroxystearic acid) type; grafted or sequential block copolymers comprising at least one block of polyorganosiloxane type and at least one block of a radical polymer; grafted or sequential block copolymers comprising at least one block of polyorganosiloxane type and at least one block of a polyether; copolymers of C₁-C₄ alkyl acrylates or methacrylates and of C₈-C₃₀ alkyl acrylates or methacrylates; grafted or sequential block copolymers comprising at least one block resulting from the polymerization of ethylenic monomers optionally comprising conjugated bonds and at least one block of a vinyl polymer; grafted or sequential block copolymers comprising at least one block resulting from the polymerization of ethylenic monomers optionally comprising conjugated bonds and at least one block of an acrylic polymer; and grafted or sequential block copolymers comprising at least one block resulting from the polymerization of dienes and at least one block of a polyether.

18. A composition according to claim 16, wherein said stabilizing agent is a grafted or sequential block copolymer comprising at least one block resulting from the polymerization of dienes and at least one block of a vinyl polymer.

19. A composition according to claim 1, further comprising at least one additional fatty phase chosen from waxes; gums; pasty fatty substances of plant, animal, mineral, synthetic or silicone origin; and mixtures thereof.

20. A composition according to claim 10, wherein said at least one coloring material comprises at least one pulverulent compound chosen from fillers, pigments and pearlescent agents.

21. A composition according to claim 20, wherein said at least one pulverulent compound is present in an amount ranging from 0% to 50% of the total weight of the composition.

22. A composition according to claim 21, wherein said at least one pulverulent compound is present in an amount ranging from 1 to 40% of the total weight of the composition.

23. A composition according to claim 1, wherein said polymer particles dispersed in the liquid fatty phase comprise dry matter present in an amount ranging from 0% to 60% of the total weight of the composition.

24. A composition according to claim 23, wherein said polymer particles dispersed in the liquid fatty phase comprise dry matter present in an amount ranging from 12 to 60% of the total weight of the composition.

25. A composition according to claim 1, wherein said liquid fatty phase comprises at least one oil chosen from C₈-C₁₆ isoparaffins, isododecane, and linear or cyclic silicones having from 2 to 7 silicon atoms, wherein said these silicones optionally comprise alkyl groups having from 1 to 10 carbon atoms.

26. A composition according to claim 1, wherein said composition is in the form of a stick, in the form of a supple paste with a dynamic viscosity at 25° C. of 1 to 40 Pa.s, in the form of a disk, of an oily gel, of an oily liquid, of a vesicular dispersion comprising ionic and/or non-ionic lipids, or in the form of a water-in-oil or oil-in-water emulsion.

27. A composition according to claim 1, wherein said composition is in an anhydrous form.

28. A composition according to claim 1, wherein said composition is in the form of a product for caring for and/or for making up the skin and/or lips.

29. A composition according to claim 28, wherein said composition is in the form of a foundation, of a blusher, of an eyeshadow, of a lipstick, of a base or balm for caring for the lips, of a product for concealing rings under the eyes, of an eyeliner, or of a mascara.

30. A process for making a composition for topical application to decrease or eliminate transfer of a film of the composition deposited on human skin or lips to a substrate brought into contact with the film or to retain its gloss, said process comprising dispersing particles of at least one polymer in a liquid fatty phase, stabilizing said particles at the surface of said liquid fatty phase by a stabilizing agent, polymerizing at least one monomer possessing an ethylenic bond to form a fat-soluble rheological agent, and thickening said fatty phase with said fat-soluble rheological agent.

31. A process for the cosmetic care of or for making up the lips or skin, said process comprising applying to the lips or skin, a cosmetic composition comprising a liquid fatty phase and polymer particles, said polymer particles being dispersed in the liquid fatty phase and stabilized at the surface by a stabilizing agent, and said liquid fatty phase being thickened by a fat-soluble rheological agent resulting from the polymerization of at least one monomer possessing an ethylenic bond.

32. A process for limiting or eliminating the transfer of a composition for making up or caring for the skin or lips onto a substrate other than the skin or lips, wherein said composition comprises a liquid fatty phase and at least one ingredient chosen from cosmetic, dermatological, hygiene and pharmaceutical active principles and coloring materials, said process comprising introducing into the liquid fatty phase of said composition a fat-soluble rheological agent resulting from the polymerization of at least one ethylenic monomer and polymer particles, which polymer particles are dispersible in said liquid fatty phase and can be stabilized at the surface of said liquid fatty phase by a stabilizing agent.

33. A composition according to claim 14, wherein said fatty esters are chosen from isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, di(2-ethylhexyl) succinate, diisostearyl malate, glyceryl triisostearate and diglyceryl triisostearate.

34. A composition according to claim 14, wherein said higher fatty acids are chosen from myristic acid, palmitic acid, stearic acid, behenic acid, oleic acid, linoleic acid, linolenic acid and isostearic acid.

35. A composition according to claim 14, wherein said higher fatty alcohols are chosen from cetanol, stearyl alcohol or oleyl alcohol, linoleyl or linolenyl alcohol, isostearyl alcohol and octyldodecanol.

36. A composition according to claim 14, wherein said silicone oils are chosen from PDMS and silicone oils which are optionally phenylated, or which are optionally substituted by aliphatic and/or aromatic groups or by functional groups.

37. A composition according to claim 36, wherein said silicone oils which are optionally phenylated are chosen from phenyltrimethicones.

38. A composition according to claim 36, wherein in said silicone oils which are optionally substituted by functional groups, said functional groups are chosen from hydroxyl, thiol and/or amine groups.

39. A composition according to claim 14, wherein said volatile oils are chosen from octamethylcyclotetrasiloxane, decamethylcyclopentasiloxane, hexadecamethylcyclohexasiloxane, heptamethylhexyltrisiloxane, heptamethyloctyltrisiloxane or C₈-C₁₆ isoparaffins, and isododecane.
